(19) United States
(12) Patent Application Publication
(10) Pub. No.: US 2023/0255668 A1
(43) Pub. Date: Aug. 17, 2023

(54) ROTATION PREVENTION MEMBER OF LEG SCREW FOR FIXING BONE

(71) Applicant: JEIL MEDICAL CORPORATION, Seoul (KR)

(72) Inventors: Chi-Hoon KIM, Goyang-si, Gyeonggi-do (KR); Jang-Hyeon SEO, Gimpo-si, Gyeonggi-do (KR); Geung-Rok KIM, Seoul (KR)

(21) Appl. No.: 18/014,512

(22) PCT Filed: Feb. 10, 2021
(86) PCT No.: PCT/KR2021/001796

§ 371 Date: Jan. 5, 2023

(30) Foreign Application Priority Data
Jul. 9, 2020 (KR) 10-2020-0084629

(51) Int. Cl. A61B 17/72; A61B 17/74; A61B 17/86

(52) U.S. Cl. CPC A61B 17/725; A61B 17/744; A61B 17/86; A61B 2017/8655; A61B 2017/00862

(57) ABSTRACT

The present disclosure relates to a rotation prevention member of a leg screw for fixing a bone that is configured to prevent rotation of a leg crew that is coupled to a nail in a medullary cavity such as a femur and fixes a fracture portion. The rotation prevention member of a leg screw for fixing a bone, in which a femoral nail is fitted in an end of a femoral marrow, a leg screw combined with a cap is laterally inserted in a transverse hole formed at an angle with respect to a longitudinal axis of the nail, and the leg screw is fixed to be longitudinally movable without rotating in the transverse hole, includes: pin guide grooves formed symmetric to each other in a longitudinal direction of the leg screw; a superelastic member inserted in the pin guide grooves; and a cap thread-fastened to a rear surface of the leg screw to prevent separation of the superelastic member in order to prevent rotation between a threaded portion of the leg screw and a bone due to rotation after a fracture part is occupied.

### TECHNICAL FIELD

The present disclosure relates to a rotation prevention member of a leg screw for fixing a bone, in more detail, the present disclosure relates to a rotation prevention member of a leg screw for fixing a bone, the rotation prevention member preventing rotation of a leg crew that is coupled to a nail in a medullary cavity such as a femur and fixes a fracture portion.

### Background Art

As the average life of human increases and old people increases with the development of medical science, patients with a fracture in a femur or a tibia increase, and firm surgical internal fixation is important in treatment for such femur fractures.

In particular, the femur also known as a thigh bone generally includes a large femur shaft continuing from a hip from a knee. The proximal end of the femur shaft includes a head, a neck, a greater trochanter, and a lesser trochanter. The head of a femur is fitted in an acetabular cup of a hip bone, thereby forming a ball and socket joint in a hip. A distal end of a femur includes a medial condyle and a lateral condyle. These condyles are engaged with the upper end of a tibia, thereby forming a knee joint. In the entirety, a femur is the longest and strongest bone of the bones of a human body. However, a femur is vulnerable to a fracture.

In old people and patients, pertrochanteric fractures are the most frequently generated in relation to fractures of the neck of a femur. Due to aging and sickly conditions that are observed in these patients, it is required to timely stabilize a damaged fracture portion in order to minimize the time that the patients spend on the bed and the time for rehabilitation. Preferably, devices and procedures that can minimize complications that are caused by a so-called immobilization syndrome are used, and these complications may be fatal to patients under a sensitive metabolic environment.

Further, it is preferable to minimize a loss of blood related to surgical procedures. Further, coupling devices that are used to enable patient to very timely take a sitting posture and take an erect posture while supporting their weight gradually within 2-3 days after a surgery should also be stable.

In general, internal fixation of a femur fracture portion is the most frequent one of orthopedic surgeries. A femur fracture is generated at both the proximal portion and the distal portion of a femur. A fracture at the proximal portion of a femur is generally classified into a femur neck fracture (condylar fracture or subcondylar fracture), an intertrochanteric fracture, and a subtrochanteric fracture. A fracture of the distal portion of a femur is called supracondylar fracture. Supracondylar fracture generally perpendicularly advances between the condyles of the lower end of a femur, thereby separating the distal portion of a femur into two main bone fragments. The fracture line is more finely divided, which may result in a plurality of small bone fragments. A fracture of a femur that advances to the neck is generally difficult to treat more than a fracture limited in the shaft of a femur.

Accordingly, an intramedullary nail (hereafter, referred to as a ‘nail’) is used to fix and stabilize a fracture of a long bone such as a femur.

An intramedullary nail may be inserted in a marrow canal of a femur and may be positioned across the fracture line in the femur. Next, a screw or another fixing member is inserted into a hole formed in the nail from the opposite side of the fractured femur, thereby fixing the opposite side of the fractured femur.

When a head or a neck of a long bone such as the head or the neck of a femur is fractured, a leg screw may be inserted into a transverse hole formed in a nail.

This hole enables a leg screw to extend to the head through the neck of a long bone across a fracture line and to reduce the fracture in the neck or the head of the long bone.

For example, as shown in FIG. 1, a shaft 12, a neck 14, and a head 16 of a medullary cavity 10 are shown.

The neck 14 of the medullary cavity 10 shown in the figure is fractured at a fracture portion 17.

As described above, a nail 20 is inserted in the fractured portion and a transverse hole 20a is formed in the nail 20. The transverse hole 20a extends through the nail 20 and has a size that can accommodate a leg screw 22. In detail, the leg screw 22 has an outer diameter slightly smaller than the transverse hole 20a. Accordingly, the leg screw 22 can reduce the fracture at the fracture portion 17 by passing through the transverse hole 20a.

However, while the bone is treated, stricture is frequency generated, so the bond is shortened at the fracture portion. Unless a femoral leg screw stops this phenomenon, there is a possibility that the leg screw breaks or the fracture portion becomes unstable in the femoral head.

In this case, there are several configurations that prevent a femoral leg screw from rotating and allow the femoral screw to longitudinally move. A femoral leg screw should not rotate with respect to a femoral iron core, whereas a fracture portion should be able to rotate with respect to the leg screw.

In order to solve these problems, according to a femoral leg screw disclosed in US-A-2002/0045900, a longitudinal slot is formed in both sides and a U-shaped locking member is pushed into the slots. This type is a combination of a screw and a blade, so the characteristics of two fasteners are combined. However, the height of branches of the locking member is relatively small, and this is because the branches are adjusted by the femoral iron core only in the slots of the leg screw, so the effect of the blade is limited.

The branches of the locking member open through the longitudinal slot of which the depth to a coupling portion gradually decreases. The locking member serves only to prevent a fracture portion from protruding from the leg screw. The method of fixing the femoral leg screw by rotating the femoral iron core is similar to that in EP-A-257, but a bolting tool is used at the end of the iron core, so a doctor has to work at both sides when inserting and fixing the leg screw.

The problem with rotation is solved by the implant system of WO-A-01/739679 in the related art, in which two end fixing screws are used.

However, there is a problem that such a surgical needs more costs and almost or completely impossible to find two screws in a small jaw or neck.

Further, the assembly of a spiral blade and a screw of US-A-2002/0045900 can be fixed not to rotate around an iron core by the element added at the end of the femoral iron core, but has a problem in that the performance of sliding in the axial direction while the screw/locking member are inserted into the femoral iron core is deteriorated, which is because when the branches of the U-shaped locking member are inserted, the branches are opened by the iron core through the slots having a small depth, whereby the branches block each other.

Accordingly, in order to solve the problems described above, an “intramedullary nail for femur fixation” has been filed and registered in Korean Patent No. 10-0755087.

This is configured such that a femoral leg screw is fitted and fixed at an angle in a side hole of a nail to be longitudinally movable without rotating in the hole, in which one or more slots are longitudinally formed in the leg screw and a locking member having one or more branches arranged parallel with the leg screw can be fitted into the longitudinal slot of the leg screw.

That is, referring to FIG. 2, in more detail, a long slot 22a is formed in both sides of a leg screw 22 and a tap hole 22b is formed at an end.

The locking member 30 has an annular ring 31 at an end and a pair of long branches 32 is connected to the ring 31.

The branches 32 are inserted through the tap hole 22b, that is, the branches 32 are inserted along the long slots 22a formed in both sides of the leg screw 22.

In this state, the leg screw is inserted into a transverse hole 20a of a nail 20.

That is, even if two long branches 32 open in the nail 20 when the leg screw 22 is longitudinally inserted, they remain inside the threads without protruding from the leg screw 22 and twist and fix a fracture portion in the leg screw 22, and the locking member 30 is prevented from separating by a cap 40.

However, since the two long branches 32 of the locking member 30 are parallel to each other, the performance of sliding in the axial direction in a surgery is deteriorated and the branches 32 are pushed out of the long slots 22a.

Further, in order to manufacture the locking member 30, it is required to separately manufacture the annular ring 31 and the long branches through pressing and then combine them through welding, so there is a problem that the cost of machining increases.

### DISCLOSURE

### Technical Problem

Accordingly, the present disclosure has been made in an effort to solve the problems in the related art described above and proposes a rotation prevention member of a leg screw for fixing a bone, the rotation prevention member preventing rotation of a leg screw due to rotation after a facture is occupied by the leg screw and a nail in a medullary cavity by coupling a superelastic pin to the leg screw that is coupled to a nail in a medullary cavity such as a femur and fixes a fracture portion.

### Technical Solution

In order to achieve the objectives of the present disclosure,

a rotation prevention member of a leg screw for fixing a bone, in which a femoral nail is fitted in an end of a femoral marrow, a leg screw combined with a cap is laterally inserted in a transverse hole formed at an angle with respect to a longitudinal axis of the nail, and the leg screw is fixed to be longitudinally movable without rotating in the transverse hole, includes: pin guide grooves formed symmetric to each other in a longitudinal direction of the leg screw; a superelastic member inserted in the pin guide grooves; and a cap thread-fastened to a rear surface of the leg screw to prevent separation of the superelastic member in order to prevent rotation between a threaded portion of the leg screw and a bone due to rotation after a fracture part is occupied.

Pin guide grooves 101 formed to be symmetrical to each other along the longitudinal direction of the lag screw 100; and

a super-elastic member 200 inserted into the pin guide groove 101; and

It consists of a cap 300 screw-fitted to the rear surface of the lag screw 100 to prevent the superelastic member 200 from coming off, and prevents rotation between the screw part of the lag screw and the bone by rotational movement after fracture reduction is configured to.

### Advantageous Effects

According to the rotation prevention member of a leg screw for fixing a bone of the present disclosure having the configuration described above, a superelastic member having a twist is installed in a leg screw that is coupled to a nail in a medullary cavity such as a femur and fixes a fracture portion, thereby being able to prevent rotation of the leg screw and firmly fix the fracture portion.

Further, according to the present disclosure, since the superelastic member has a thin pin shape, so manufacturing is simple. Further, since there is no need for pressing and welding, the cost can be reduced and the production efficiency can be increased.

### DESCRIPTION OF DRAWINGS

FIG. 1 is a cross-sectional view showing a portion of a nail of the related art positioned in a femur and a leg screw disposed through a transverse hole of the nail to attenuate a fracture in a femoral neck.

FIG. 2 is a view showing a surgery for a fracture using a locking member of the related art fitted in a longitudinal slot of a leg screw.

FIG. 3 is an exploded perspective view of a rotation prevention member of a leg screw for fixing a bone according to the present disclosure.

FIGS. 4A to 4C are views showing a process of assembling the rotation prevention member of a leg screw for fixing a bone, in which

FIG. 4A is a view showing the state before a superelastic member is coupled to a leg screw,

FIG. 4B is a view showing the state in which the superelastic member is partially inserted in pin guide grooves of the leg screw, and FIG. 4C is a view showing the state in which a cap is coupled with the superelastic member fully inserted in the leg screw.

FIG. 5 is an enlarged view showing main parts when the superelastic member and the cap are coupled to the leg screw according to the present disclosure.

FIG. 6 is a cross-sectional view showing the state in which a leg screw has been implanted in a medullary cavity and then the rotation prevention member of the present disclosure has been implanted.

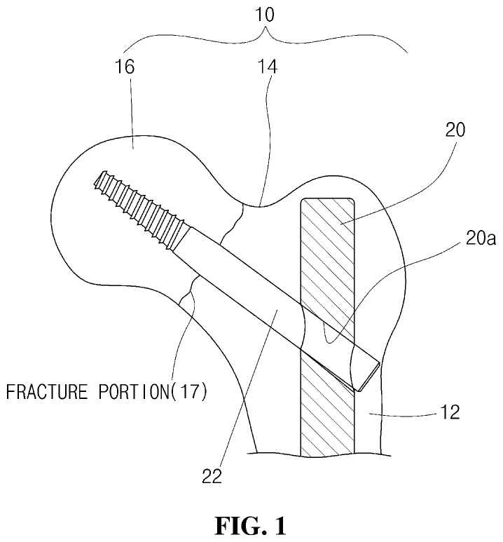

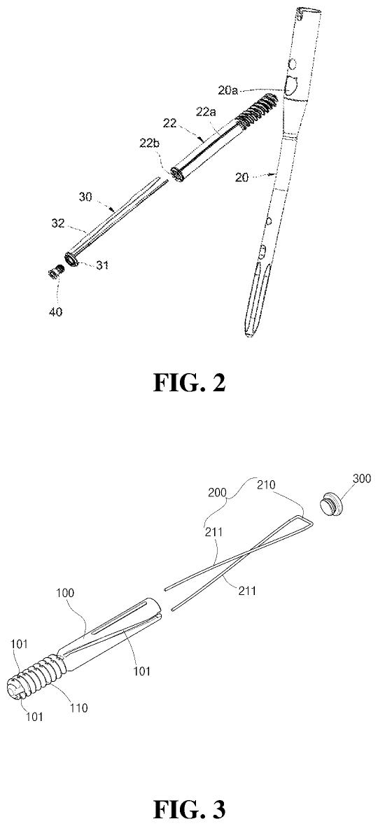

### BEST MODE

Hereafter, the configuration of a rotation prevention member of a leg screw for fixing a bone of the present disclosure is described in detail with reference to the drawings.

However, the disclosed drawings are provided as examples so that the spirit of the present disclosure can be sufficiently transmitted to those skilled in the art. Accordingly, the present disclosure is not limited to the proposed drawings and may be implemented by other ways.

Further, unless stated otherwise, the terms used herein have meanings that those skilled in the art generally understand, and well-known functions and configurations that may make the main idea of the present disclosure unclear in the following description and the drawings are not described in detail.

The present disclosure, in which a femoral nail is fitted in an end of a femoral marrow, a leg screw combined with a cap is laterally inserted in a transverse hole formed at an angle with respect to the longitudinal axis of the nail, and the leg screw is fixed to be longitudinally movable without rotating in the transverse hole, as shown in FIG. 3, is composed of pin guide grooves 101 formed on a leg screw 100, a superelastic member 200, and a cap 300.

In more detail, the leg screw 100 is formed in the same way as the related art, but is different in that symmetric spiral pin guide grooves 101 are formed on both sides thereof, respectively.

In addition, the pin guide grooves 101 are formed throughout the body of the leg screw 100 to a threaded portion 110.

A connecting rod 210 having a thin pin shape is formed at a first end of the superelastic member 200 that is inserted in the pin guide grooves 101, and a pair of elastic pins 211 integrally bends from both ends of the connecting rod 210.

The superelastic member 200 has pseudoelasticity or superelasticity and uses the property of a shape-memory alloy returning into a preset shape.

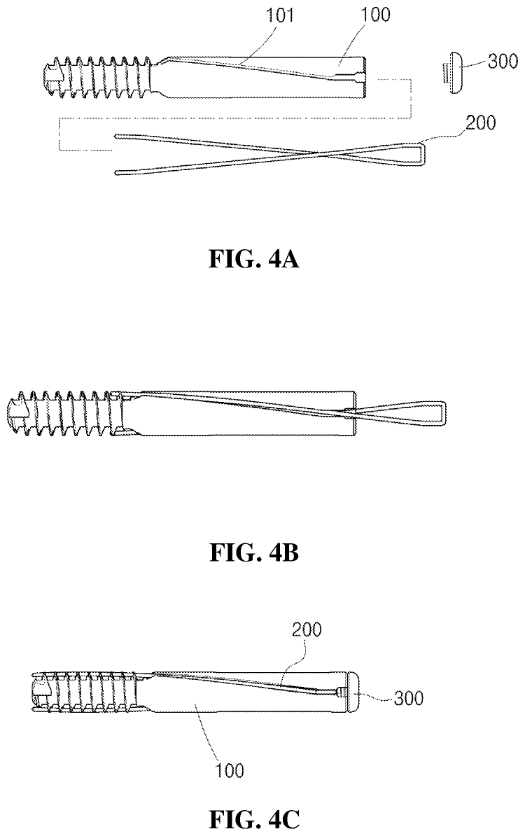

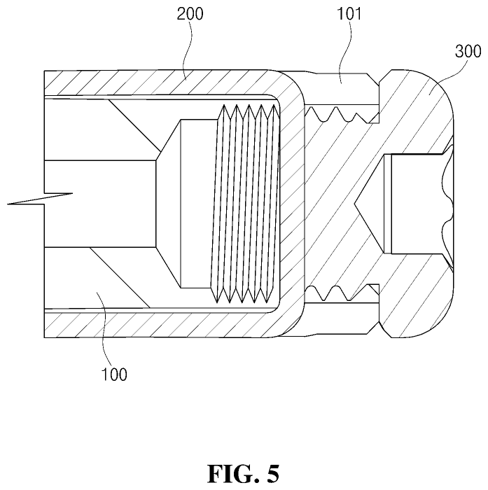

In addition, the cap 300, as shown in FIG. 4c, is thread-fastened to the rear end of the leg screw 100 while pressing the connecting rod 210 of the superelastic member 200.

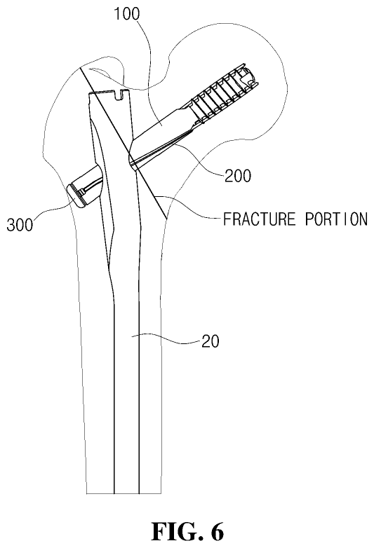

Accordingly, a rotation prevention member 1 of a leg screw for fixing a bone having this configuration, as shown in FIG. 6, uses the nail 20 and the leg screw to treat a fracture portion 17 of a medullary cavity 10 in a surgery, which is the same as the related art.

However, according to the present disclosure, the superelastic member 200 is fitted in the leg screw 100 that is coupled to the nail 20 and fixes the fracture portion 17 in the medullary cavity to prevent the leg screw 100 from rotating.

In more detail, as shown in FIG. 4A, with the leg screw 100, the superelastic member 200, and the cap 300 prepared, as shown in FIG. 4B, the superelastic member 200 is fitted into the pin guide grooves 101 of the leg screw 100, and then assembled, as shown in FIG. 4C.

Next, as shown in FIG. 6, the leg screw 100 combined with the superelastic member 200 is pushed into the transverse hole 20a formed in the nail 20, thereby being coupled to the fracture portion.

Accordingly, while the leg screw 100 is fastened to the fracture portion, the superelastic member 200 opens and comes in close contact with the fracture portion due to elasticity.

Accordingly, the superelastic member 200 firmly presses the leg screw 100, thereby being able to prevent rotation of the leg screw.

Further, although the configuration and rotation of a rotation prevention member of a leg screw for fixing a bone of the present disclosure were described above, the present disclosure may be changed, modified, and replaced in various ways by those skilled in the art, and the change, modification, and replacement should be construed as being included in the protective range of the present disclosure.

### DESCRIPTION OF THE REFERENCE NUMERALS

| | |
|---|---|
| 10: leg screw | 101: pin guide groove |
| 110: threaded portion | 200: superelastic member |
| 210: connecting rod | 211, 211: elastic member |
| 300: cap | |

1. A rotation prevention member of a leg screw for fixing a bone, in which a femoral nail is fitted in an end of a femoral marrow, a leg screw combined with a cap is laterally inserted in a transverse hole formed at an angle with respect to a longitudinal axis of the nail, and the leg screw is fixed to be longitudinally movable without rotating in the transverse hole, the rotation prevention member comprising:
a pin guide grooves formed symmetric to each other in a longitudinal direction of the leg screw;
a superelastic member inserted in the pin guide grooves; and
a cap thread-fastened to a rear surface of the leg screw to prevent separation of the superelastic member in order to prevent rotation between a threaded portion of the leg screw and a bone due to rotation after a fracture part is occupied.

2. The rotation prevention member of claim 1, wherein the pin guide grooves are formed on a body to which the cap is coupled, and the threaded portion.

3. The rotation prevention member of claim 1, wherein the pin guide grooves are formed a long spiral shape.

4. The rotation prevention member of claim 2, wherein the superelastic member has Pseudoelasticity or superelasticity.

5. The rotation prevention member of claim 2, wherein the superelastic member has a connecting rod formed in a thin pin shape at a first end thereof, and elastic pins integrally bend from both ends of the connecting rod, respectively.
